(19) United States
(12) Patent Application Publication
(10) Pub. No.: US 2009/0178282 A1
(43) Pub. Date: Jul. 16, 2009

(54) Hair Removal With Fluid Delivery

(76) Inventors: Gordon Gerald Guay, Chelmsford, MA (US); Sonia Rubico-Jamir, Andover, MA (US); Frank Beerwerth, Kaltenholzhausen (DE); Uwe Bielfeldt, Bad Soden Am (DE); Bernhard Kraus, Braunfels (DE); Robert Jung, Friedberg (DE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/350,446

(22) Filed: Jan. 8, 2009

Related U.S. Application Data
(60) Provisional application No. 61/010,863, filed on Jan. 11, 2008.

(51) Int. Cl. B26B 19/40; A61B 18/20

(52) U.S. Cl. 30/41.5; 606/9

(57) ABSTRACT

A hair removal system includes a hair removal device with a hair removal area such as a razor cartridge, a dry shaving hair removal area, an epilating area, or a laser hair removal area, each having a fluid delivery system with at least one gradient foam having a fluid and a applicator surface area open to the environment such that the fluid is passively dispensed onto a skin surface independent of gravity through said applicator surface area while a user is shaving or epilating. The fluid delivery system may be integrated via pins, glue or other locking mechanism onto the hair removal area (e.g. razor cartridge). A cap covers the gradient foam to prevent the foam from drying out. A fluid reservoir may be integrated in the hair removal system in the hair removal area or in a handle. The fluid reservoir serves to replenish fluid in the foam. The fluid may be any oil, soap, moisturizer or other suitable liquid for shave preparation or for providing post hair management related benefits. The hair removal device may be a razor, a dry shaver, an epilator or a laser (light based system).

### CROSS REFERENCE TO RELATED APPLICATION

This application claims the benefit of U.S. Provisional Application No. 61/010,863 filed Jan. 11, 2008.

### FIELD OF THE INVENTION

The field of the invention is fluid delivery systems and more particularly, a hair removal device having an integrated fluid delivery system.

### BACKGROUND OF THE INVENTION

When a user is removing hair from their skin, for instance in wet shaving, a user typically “shave-preps” or prepares the area of skin (or hair) to be shaved by lubricating or preparing the area with a fluid or lotion to provide a safe and close shave, generally spreading the fluid around with their hands. In shaving, a razor is used to shave the lubricated hairs off the skin in the shave-prepped area. Hence, this is generally a two-step process for each area desired to be shaved. Further, a user may have to wash off their hands after applying the preparatory fluid before the second shaving step so as to hold the razor properly.

There are a variety of known methods that exist today to deliver fluids, many where fluids do not come into direct contact with one's hands. Within those, there are three broad prior art categories of fluid delivery types. One prior art category would include mechanical systems, such as brushes, pads, and foams. A drawback of these systems is the lack of consistency in fluid output and the need to continuously refill. The second category would include pressurized or electromagnetic driven systems which are generally active. By being active, these systems are always on or require energy to operate, thereby making them relatively costly solutions for desired low cost or mass marketed products.

A third category of fluid delivery systems would include capillary (or wick) based systems which are generally passive. Prior art capillary systems would include sintered powders, filamentous, foam or fiber based systems.

Some common examples of capillary system products would be wick-based air freshener fragrance-delivery products or some ink jet printer delivery products. The fiber based systems use bundles or other structures that are physically attached to each other. Some examples of these are: felt tip pens, magic markers, porous dome applicators (e.g. sintered pressed powder). However, as with other prior art solutions, fiber bundles are not necessarily consistent in the amount of product delivered and attempts to solve that problem by varying the applicator size adds difficulty. Furthermore, multiple uses of these systems can result in cloggage over time rendering inconsistent delivery of material to the desired dose.

The gradient or compressed foam described for fuel cells in U.S. Pat. No. 6,994,932, requires both a pump and capillary-based system, increasing the cost and complexity of the final device. Additionally, a limitation of the system described therein is that in its application to fuel cells for electronic devices it is a self-contained system, i.e. not open to the environment due to volatility of the fuel fluid.

Although these prior art systems described above function properly, some have inconsistent fluid delivery, some require energy, some are closed to the environment and there can be great variability in fluid delivery due to manufacturing, inconsistencies from product to product, and/or changes within a given product over time.

Further, these prior art systems are limited as they do not provide a fluid delivery system simplifying use with a wet or dry hair removal device such as a shaver or an epilator, while also providing continuous, tailored and precise fluid delivery nor the capability of using a varying number and type of fluids, such as aqueous or non-aqueous (or oil) based fluids.

A need exists in hair removal or shaving for a user to be able to apply a fluid without requiring a user's hands to spread a shave prep fluid or a fluid for providing post hair management related benefits onto the user's skin and to save a step in the shave process while also delivering fluids consistently, passively, independently of orientation and gravity, open to the environment and with the ability to tailor the amount of fluid delivered to a unit of area, thereby reducing cost and increasing effectiveness.

A further need exists to deliver any type of fluid or a plurality of fluids or formulations directly while also being integrated with a hair removal device such as a razor, a dry shaving device or an epilator or laser for use on both wet and dry skin.

### SUMMARY OF THE INVENTION

In one aspect, the invention features, in general, a hair removal device including a hair removal area having at least one gradient foam, each gradient foam having an applicator surface area open to the environment, wherein the gradient foam is filled with a fluid which is passively and consistently dispensed independent of gravity when the applicator surface area contacts a skin surface. The hair removal area can be a razor cartridge having an over-frame and razor blades, a dry shaving hair removal area, an epilating area, or a laser hair removal area.

In another aspect, the gradient foam does not extend past tips of the razor blades and substantially covers the over-frame. In another aspect, the gradient foam has a varying compression ratio in the range of about 0 to about 20 over its length. In still other aspects of the invention, the gradient foam is comprised of polyurethane, melamine, cellulosic, PVC, polystyrene, polyethylene, or polyester materials and may be formed by a composite of a plurality of foams having different compression ratios.

Particular embodiments of the invention include one or more of the following features. In one particular embodiment, the gradient foam is chemically modified. In another embodiment, a first gradient foam has a first fluid and a second gradient foam has a second fluid wherein the first and second fluids are of a different type and the first and second gradient foams have the same compression ratios. In a alternate embodiment, there is a first fluid within a first gradient foam and a second fluid within a second gradient foam, but the first and second fluids are of a different type and said first and second gradient foams have different compression ratios. The first and second fluids can interact to form a third component that is applied onto the skin surface. In a still further embodiment, at least one fluid reservoir is in contact with the at least one gradient foam. The fluid reservoir can be in a handle of the hair removal device or in the hair removal area. The gradient foam is preferably locked into said hair removal area. A cap may be included to cover the gradient foam and can be made of a plastic injected polymer material. The fluid is preferably a liquid having at least one ingredient and a viscosity of in the range of about 0.1 to about 2000 centipoise. The applicator surface area may have a domed shape. In addition, as the applicator surface area increases, the amount of fluid dispensed onto the surface increases in a directly proportional manner.

In another aspect, the invention describes a method of delivering fluid through a hair removal device, includes lubricating a user's skin surface, with a fluid flowing upon contact with the skin surface from a applicator surface area of at least one gradient foam held in a hair removal area and removing hair with the hair removal device from the user's skin surface, wherein hands of the user are not in contact with the fluid and wherein the lubricating and shaving steps occur substantially simultaneously. The hair removal area can be one of the following: a razor cartridge, a dry shaving hair removal area, an epilating area, or a laser hair removal area.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram of a prior art wedge of wicking material prior to compression.

FIG. 2 is a schematic diagram of the wedge of wicking material of FIG. 1 after compression.

FIG. 3 is a diagram of a razor with two gradient foams in accordance with the present invention.

FIG. 4 is a diagram of a razor with a gradient foam structure in accordance with the present invention.

FIG. 5 is a side-view of the razor in FIG. 3 in accordance with the present invention.

FIG. 6 is a diagram of FIG. 3 showing a reservoir in accordance with an alternate embodiment of the present invention.

FIG. 7 is a diagram of FIG. 3 showing a cap in accordance with a preferred embodiment of the present invention.

FIG. 8 is a diagram of a dry shaver with a gradient foam structure in accordance with a preferred alternate embodiment of the present invention.

FIG. 9 is diagram of an epilator with a gradient foam structure in accordance with a preferred alternate embodiment of the present invention.

FIG. 10 is a graph depicting the relationship between the applicator surface area and average fluid released.

### DETAILED DESCRIPTION OF THE INVENTION

For purposes of the present invention, the term “fluid” will be defined as a substance such as a liquid that is capable of flowing and that changes its shape at a steady rate when acted upon by a force tending to change its shape and may be used interchangeably or signify the following terms: liquid, water, alcohol, silicone, oil, emulsion or any combination of these fluid terms or with any additional soluble components.

Fluids desired in the present invention are of a type that produce low viscosity formulations with the capability of flowing within a given pore structure where viscosity is low enough to function via capillary action and does not require any pressure for delivery.

Additionally, the term “wicking” and “capillary action” are deemed equivalent for purposes of the present invention and can be used interchangeably.

The term “hair removal device” herein signifies a wet and/or dry shaving system which includes a razor, a dry shaver, an epilator, or a laser (light-based system) having respective hair removal areas, a razor cartridge, a dry shaver hair removal area, an epilating area or a laser hair removal area.

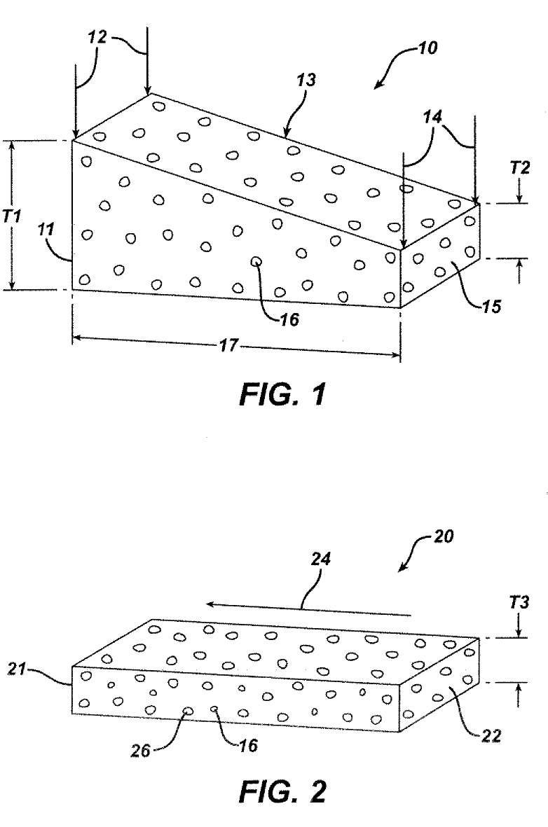

Referring now to FIG. 1 shows a prior art wedge of foam or wicking material prior to felting. Wicking is generally known to mean the drawing off of a fluid by capillary action. As described in “Fluid Fuel Reservoir for Fuel Cells”, U.S. Pat. No. 6,994,932, the wicking structure therein is made with a foam with a capillary gradient, or gradient foam, such that the flow of fluid fuel is directed from one region of the structure to another region as a result of the differential in capillarity between the two regions, where the direction of capillary flow of fluid is from a lower capillarity region to a higher capillarity region, or otherwise stated from low to high density regions of the foam.

One method for producing a material with a capillary gradient is to “felt,” or compress, or heat set, foam to varying degrees of compression or pore size along its length. Another method for producing a material with a capillary gradient is to assemble a composite of individual components with distinctly different capillarities (or compression ratios) that are physically attached to each other.

A piece of foam or wicking material is generally known to have many pores generally of consistent size and typically described as having a certain number of pores per square inch (ppi). PPI designations are generally used for foams. It follows that the more pores (per unit area) that exist in the foam, the smaller the pores are, thereby rendering the foam less porous. The fewer pores there are in the foam, the bigger the pore size, thereby rendering the foam more porous. The pore sizes in a piece of foam with consistent pore size can be made to vary as will be described below with respect to FIGS. 1 and 2. By varying the pore size in a piece of foam (i.e. changing the gradient of porosity), one side of the foam will be more porous than the other. Therefore, any fluid placed in the foam will trend from the more porous area (largest pores) toward the least porous area (smallest compressed pores).

When open cell foams are manufactured they have “windows” of a thin layer of material within the general foam structure. These windows are typically removed either chemically or via a zapping process with a flash flame to produce an open cell structure described as reticulated foam. It is desirable to produce gradient foams using foam that has been reticulated.

FIGS. 1 and 2 illustrate the before and after schematics of a wicking material, such as foam, with a capillary gradient.

As shown in FIG. 1, a wedge-shaped piece 10 of foam of consistent density and consistent pore size has a first thickness T1 at a first end 11 and a second thickness T2 at a second end 15. The foam 10 has a predefined starting material and porosity and is physically cut at an angle to form the wedge-shape as shown in FIG. 1. Foam piece 10 has evenly spaced pores 16 and consistent pore size, with initial pore size of each pore 16 preferably ranging from about 20 pores per square inch (ppi) to about 120 ppi. The angle of gradient varies with the thicknesses T1 and T2, and the length of foam piece 17.

Foam piece 10 is preferably made of polyurethane but can be made from, though not limited to, any of the following materials: melamine, cellulosic, PVC, polystyrene, polyethylene, and polyester. The material of the foam piece can vary and can be surface treated to achieve other surface energy properties. For instance, the foam piece 10 can be surface treated with a chemical to increase hydrophobicity (i.e. having less affinity for water) or the starting foam material can be hydrophobic in nature.

To produce a material with a capillary gradient, one may subject the foam to varying degrees of compression along its length. Another way is to assemble together several different pieces of reticulated foam having different pore sizes with different capillarities which are laminated together to create a composite material.

To accomplish the compression, the piece of foam 10 is subjected to a felting step which involves high temperature compression for a desired time thereby compressing the foam piece 10 to a consistent thickness T3, which is preferably a thickness less than or equal to T2 and less than the thickness T1 as depicted in FIG. 2. The foam produced from foam piece 10 after this type of compression is depicted in FIG. 2 and is herein referred to as gradient foam 20.

It should be noted that a greater compressive force, represented by arrows 12 in FIG. 1, is required to compress the material from T1 to T3 at the first end 11 than is the compressive force, represented by arrows 14 in FIG. 1 required to compress the material from T2 to T3 at the second end 15.

When the foam 10 is compressed, the pore sizes in foam 20 are also compressed as depicted in FIG. 2, because the construction or structure around the pores 16, i.e. the voids 26, has been compressed or destroyed. This results because the compression causes the collapse of the structures around the pores and in many instances the “compressed pores” may not look as they did in their pre-compression state, but they may not be necessarily smaller. This visual effect is a product of the smaller voids because the greater the compression, the smaller the voids over the length of the foam. It should be noted that some pores may remain intact after compression. For purposes of description herein the term “compressed pores” is generally meant to signify both the pores 16 and the voids 26 that are produced in the gradient foam FIG. 2.

The compression ratio (T1/T2) of the foam material preferably ranges from about zero (0) to about twenty (20) for gradient foam 20. The compression varies along the length of the felted or gradient foam 20 shown in FIG. 2, with the greatest compression having occurred at the first end 21 (T1 to T3) as compared with the second end 22 (T2 to T3).

Accordingly, after compression, the compressed pores 16 and 26 are smaller at the first end 21 than at the second end 22. The compression ratio of compressed pores 16 and 26 vary from about zero (0) at the first end 21 to about twenty (20) times at the second end 22.

The capillary action or capillarity is inversely proportional to the effective capillary radius, and the effective capillary radius decreases with increasing firmness or compression. Accordingly, the fluid will flow to the hardest part (or the most compressed portion of the gradient structure) of the gradient foam.

Arrow 24 in FIG. 2 represents the direction of capillary flow from the region of lower felt firmness or capillarity to higher felt firmness or capillarity. Thus, if a wicking material or wicking structure is formed with a material or composite material having a particular capillary gradient, any fluid wicked into the material may be directed to flow as shown by arrow 24, from one region of the material with lower compression ratio to another region with higher compression ratio, or from the bigger compressed pore sized end to the smaller compressed sized end. Because of the compressed pores in the gradient foam 20, this fluid flow is accomplished passively and independent of gravity.

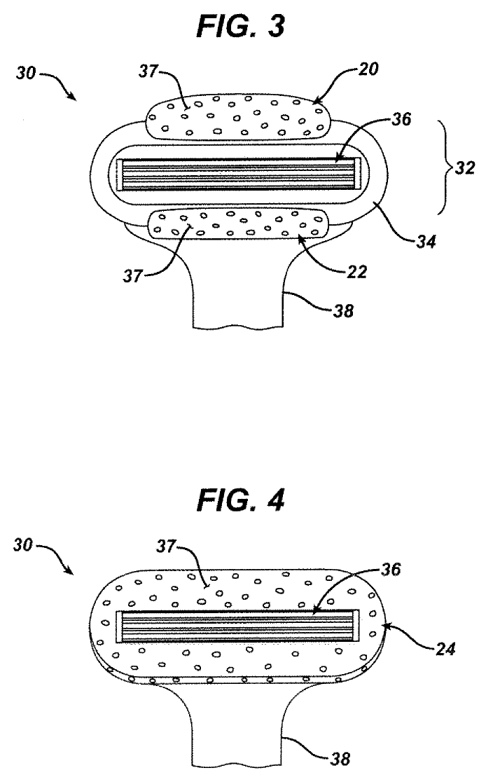

Referring now to FIG. 3, in accordance with a preferred embodiment of the present invention, a razor 30 is shown to include the gradient foams 20 and 22 as depicted in FIG. 2 on either side of a hair removal area or razor cartridge 32 and open to the environment. The razor can include one or more gradient foams and the foams may have the same or different gradient of porosities and may contain the same or different fluid ingredients or formulations.

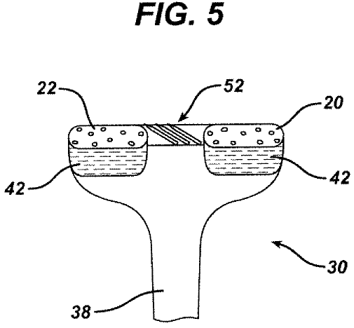

Cartridge 32 preferably has an over-frame 34 surrounding razor blades 36. The gradient foams 20 and 22 are preferably held or locked in place with pins on both sides of the over-frame 34 or snapped or clipped on. The gradient foams 20 and 22 preferably are coupled with a holder (not shown) which physically attaches to the cartridge 32 or alternatively, the foams may be attached directly to the cartridge 32. The gradient foams 20 and 22 may also be glued onto the over-frame 34 using water resistant glue or may be inserted physically into the over-frame 34 through slots or openings (not shown). Any mechanism to hold or lock the foams 20 and 22 in place may be utilized taking into consideration the expansion of the gradient foams 20 and 22 upon wetting. Preferably, as shown in FIG. 5, applicator surface areas 37 of foams 20 and 22 do not extend above or past the blade tip height 52 of the razor blades 36 such that the razor blades 36 of razor 30 are able to effectively contact a user's skin so that a user can shave their hair.

The over-frame 34 may also be substantially covered by a gradient foam material 24 as shown in FIG. 4. As can be seen, the gradient foam 24 in this alternate embodiment essentially surrounds the circumference of the razor blades 36 but preferably does not extend above or past the blade tip height 52 of the razor blades 36 as shown in FIG. 5 such that the razor 30 is able to effectively contact a user's skin so that a user can shave their hair.

A user will be able to hold handle 38 and shave with razor 30 using a similar motion over the skin as with any other razor with a handle. The gradient foams 20, 22 and 24 in FIGS. 3 and 4, respectively, are filled or wicked with fluid such as an oil, soap or shave gel or any other formulation desired for skin smoothness or shave preparation or for providing post hair management related benefits and the fluid delivery will flow, based on capillarity, out of the gradient foams 20 from applicator surface area 37 when put in contact with the skin. The fluid wicked into the gradient foams need not be necessarily the same in both foams 20 and 22 shown in FIG. 3. For instance, gradient foam 20 in FIG. 3 may contain a simple soap composition while gradient foam 22 may contain a moisturizer composition. Furthermore, it is contemplated that the fluids or ingredients of foam 20 may interact with the fluids or ingredients of foam 22 to react or form a third fluid ingredient or component to be applied onto the skin surface.

Nonetheless, the fluid flows to the applicator surface area 37 of gradient foams 20, 22, and 24 with no pump, pressure or other active mechanism in accordance with a preferred aspect of the present invention. A slight contact with the skin surface is all that is needed for fluid flow.

The razor 30 in the present invention, mated with the gradient foams filled with fluid, allows the user to have an all-in-one device with one shaving step rather than two, in that the user is able to lubricate the skin with fluid substantially simultaneously with shaving and without requiring direct hand contact with the fluid.

Applicator surface area 37 of gradient foams 20 and 22 preferably has a dome-like shape as shown in FIG. 3 but may be a flatter shape as shown in FIG. 4 or any other shape desired. The applicator surface area 37 can be changed or sculpted for any desired shape and may have protruding, rounded or beveled nubs on the surface 37 or any type of different edges, allowing the exposed applicator surface area 37 to be tailored for any required fluid release.

The fluid in gradient foam 20 may empty with use or dry out or evaporate over time. This time depends on how much fluid was initially loaded into the foam 20. When the majority of the fluid in gradient foam 20 runs out or dries out, the “empty” gradient foam 20 can be removed and replaced with new foam or a new cartridge 32 with a new foam 20 where the foam is pre-loaded with fluid, or the “empty” gradient foam 20 can be refilled with fluid.

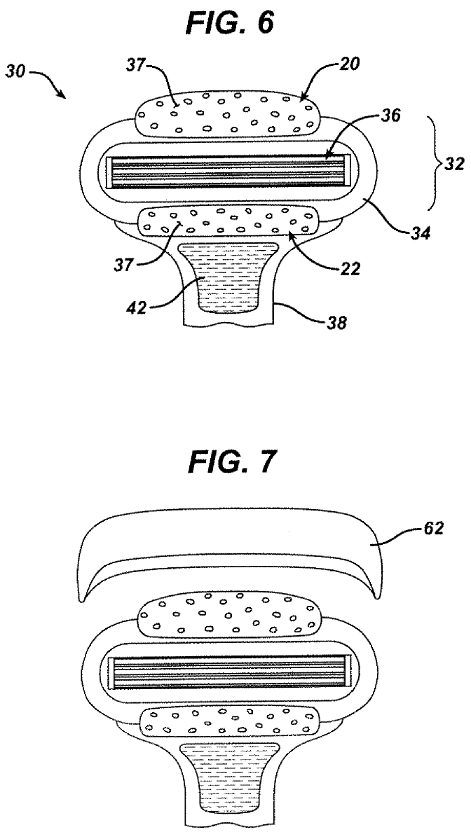

Furthermore, razor 30 may include one or more fluid reservoirs 42 within the razor 30 and preferably in contact or proximal to the gradient foams 20 and 22; for instance, underneath or behind the bottom of foams as shown in FIG. 5. One or more fluid reservoirs 42 may also be located inside the handle 38 as shown in FIG. 6. The reservoir 42 may be coupled to the handle 38 via a snap closure. The fluid reservoir 42 provides additional fluid for the gradient foams 20 and 22 in razor 30 to replenish the fluid in gradient foam 20 after it runs out or dries out.

Hence, razor 30 may be treated as a disposable razor, having a single use or multiple uses depending on the amount of fluid in the foam and the desired or potential number of uses of the replaceable with razor cartridge 32. Accordingly, the gradient foams themselves may be disposable and replaceable with new gradient foams or alternatively, the razor cartridge 32 including the foams may together be disposable and replaceable with a new razor cartridge having new gradient foams. The fluid reservoirs 42 may also be replaceable such that if the fluid in the reservoir is empty a new fully-filled reservoir 42 may be snapped in to the handle or added together with new foams.

The foam material of gradient foam 20, 22, 24 is preferably made of polyurethane but can be made from, though not limited to, any of the following materials: melamine, cellulosic, PVC, polystyrene, polyethylene, or polyester. Furthermore, foam 20 can be a single contiguous foam material with varying compressed pore sizes (i.e. continuous gradient of porosity) as shown in FIG. 2 or foam 20 can be a laminate or a composite of several foams (not shown), each having different capillarities or compression ratios, thereby producing a gradient of laminated foam porosities.

The foam 20 can be any color, shape or have any varying size of pores desired for a specific application. Preferably, the starting range of pore size for gradient foam 20 before compression is generally from about 20 ppi to about 120 ppi.

Accordingly, to provide maximum shaving comfort along with other benefits, any optimization of the gradient foams including modifications to the applicator surface area, foam density, hardness, geometry/size, foam distribution or orientation in or around the cartridge and foam locking design is contemplated in alternate embodiments of the present invention.

Furthermore, razor 30 with mated gradient foam 20 may also be attachable to any other device or surface, such as a convenient shower wall fixture or other type of docking station.

Cap 62, as shown in a preferred embodiment of the present invention in FIG. 7, covering the applicator surface area 37, circumvents the acceleration of the gradient foam 20 drying out when not in use. In some instances, the fluid will not dry out readily (i.e. oil-based formulation) and a cap or cover may or may not be necessary. In an alternate embodiment of the present invention, there is no cap but rather a nonvolatile formulation within the foam or reservoir that assists in preventing fluids from drying out. The cap 62 is preferably made of a plastic material, such as but not limited to, injected plastic polymer material (e.g. polypropylene).

It is contemplated in the present invention that the fluid or fluids in the gradient foam can be of any substance, such as a liquid that is capable of flowing. As mentioned above, the fluid is wicked into or fills up the gradient foam 20. The fluid can be aqueous or non-aqueous, having from at least one ingredient or components to many complex ingredients. The fluid in the gradient needs to be “flowable,” that is, it should have a viscosity low enough to allow the fluid to freely move through the foam. The more viscous (e.g. gel or cream) the fluid, the less likely the fluid will move through the foam without the need of pressure to push it through the pores. Accordingly, the viscosity for fluids used in the present invention preferably ranges from about 0.1 to about 2000 centipoise. Additionally, to allow proper flow for aqueous and non-aqueous fluids, empirical determinations need to be made for specific chemistries and surface energy profiles of the foam and/or fluid. And it further may be necessary, in order to achieve proper delivery for a given fluid, to modify the foam 20 chemically or via plasma-etching.

Thus, the gradient foam 20 can be modified to deliver materials with different properties such as hydrophobic or hydrophilic materials. The fluid composition in the present invention, as mentioned above, may include soap, moisturizers, active skin or hair therapeutics, anti-bacteria actives or aroma ingredients or any combinations thereof for desired benefits. The amount of fluid in the foam may vary.

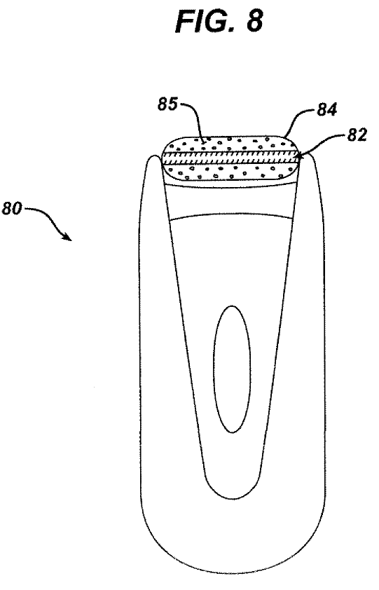

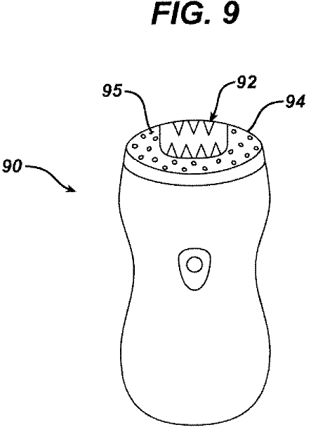

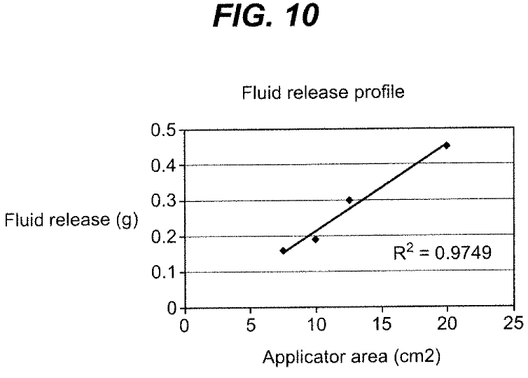

In yet another preferred embodiment of the present invention, gradient foam may be mated (in a similar fashion as described supra with respect to a razor) to a dry shaving system, which may be an electric razor, as shown in FIG. 8 or still yet to an epilator system as shown in FIG. 9.

Referring now to FIG. 8, dry shaver 80 is shown to include gradient foam 84 surrounding the dry shaver hair removal area 82 of dry shaver 80. Similarly, with respect to FIG. 9, an epilator 90 is shown to include gradient foam 94 surrounding the epilating area 92 of epilator 90. The shaver 80 and epilator 90 can include one or more gradient foams in any arrangement. When using a plurality of foams, the foams may have the same or different gradient of porosities and may contain the same or different fluid ingredients or formulations. Gradient foams 84 and 94 have applicator surface areas 85 and 95, respectively. Gradient foams 84 and 94 of shaver 80 and epilator 90 function in the same manner and have the same properties and characteristics and can be implemented in similar embodiments as described above in conjunction with FIGS. 3-7. Though not shown, similarly, a laser (light based device) can include gradient foams as described above.

It should be noted that in each of the razor, dry shaver, and epilator embodiments, the gradient foam is attached at or near the top of the device (for instance near the cartridge or other hair removal areas) thereby eliminating a user's second step of having to apply fluid during hair removal (i.e. the lubrication and shaving occur substantially simultaneously) and furthermore, the hands of the user are not in contact with said fluid flowing from the gradient foam.

The term, therefore, for the general area where the gradient foam is located, regardless of embodiment, is near the hair removal area and the hair removal area may encompass a razor cartridge, a dry shaver hair removal area or an epilating area.

The overall relationship of the applicator surface area (37, 85 and 95) versus the average fluid released from a gradient foam such as those described above in conjunction with the preferred embodiments of the present invention is shown in the graph of FIG. 10. As depicted, there is a substantially linear relationship that results, such that, as the applicator surface area increases, the amount of fluid released increases in a directly proportional manner. It is important to note that the data depicted in the graph of FIG. 10 is empirically determined for a specific formulation and specific gradient foam, the dose delivered on a skin surface at a specific application speed (e.g. the speed of the razor moving across the skin).

These results (i.e. for a given formulation, increasing the applicator surface area increases the dose in a linear fashion) will generally hold true for a given type of fluid or formulation delivered from a given type of compressed or gradient foam. In other words, it is expected to achieve the same dose profile per unit area, per formulation, speed of application and type of applicator surface area.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair removal device comprising:
a hair removal area having at least one gradient foam, each said at least one gradient foam having an applicator surface area open to the environment, wherein said at least one gradient foam is filled with a fluid which is passively and consistently dispensed independent of gravity when said applicator surface area contacts a skin surface.

2. The device of claim 1 wherein said hair removal area is a razor cartridge having an over-frame and razor blades.

3. The device of claim 1 wherein said hair removal area is an epilating area.

4. The device of claim 1 wherein said hair removal area is a dry shaving hair removal area.

5. The device of claim 2 wherein said at least one gradient foam does not extend past tips of said razor blades.

6. The device of claim 2 wherein said at least one gradient foam substantially covers said over-frame.

7. The device of claim 1 wherein said at least one gradient foam has a varying compression ratio in the range of about 0 to about 20 over a length of said at least one gradient foam.

8. The device of claim 1 wherein said at least one gradient foam is comprised of polyurethane, melamine, cellulosic, PVC, polystyrene, polyethylene, or polyester materials.

9. The device of claim 1 wherein said at least one gradient foam is formed by a composite of a plurality of foams having different compression ratios.

10. The device of claim 1 wherein said at least one gradient foam is chemically modified.

11. The device of claim 1 further comprising a first fluid within a first gradient foam and a second fluid within a second gradient foam wherein said first and second fluids are of a different type and said first and second gradient foams have the same compression ratios.

12. The device of claim 1 further comprising a first fluid within a first gradient foam and a second fluid within a second gradient foam wherein said first and second fluids are of a different type and said first and second gradient foams have different compression ratios.

13. The device of claim 11 or 12 where said first and second fluids interact to form a third component that is applied onto said skin surface.

14. The device of claim 1 wherein said hair removal device further comprises at least one fluid reservoir in contact with said at least one gradient foam.

15. The device of claim 12 wherein said at least one fluid reservoir is in a handle of said hair removal system.

16. The device of claim 12 wherein said at least one fluid reservoir is in said hair removal area.

17. The device of claim 1 wherein said at least one gradient foam is locked into said hair removal area.

18. The device of claim 1 further comprising a cap to cover said at least one gradient foam.

19. The device of claim 16 wherein said cap is comprised of a plastic injected polymer material.

20. The device of claim 1 wherein said fluid is a liquid having at least one ingredient.

21. The device of claim 1 wherein a viscosity of said fluid is in the range of about 0.1 to about 2000 centipoise.

22. The device of claim 1 wherein said applicator surface area has a domed shape.

23. The device of claim 1 wherein as the applicator surface area increases, the amount of fluid dispensed onto said skin surface increases in a directly proportional manner.

24. A method of delivering fluid through a hair removal device, comprising the steps of:
lubricating a user's skin surface, with a fluid flowing upon contact with said skin surface from a applicator surface area of at least one gradient foam held in a hair removal area; and
removing hair with said hair removal device from said user's skin surface, wherein hands of said user are not in contact with said fluid and wherein said lubricating and shaving steps occur substantially simultaneously.

25. The method of claim 24, wherein said hair removal area is one of the following: a razor cartridge, a dry shaving hair removal area, an epilating area, or a laser hair removal area.
